(19) United States
(12) Patent Application Publication
(10) Pub. No.: US 2020/0046212 A1
(43) Pub. Date: Feb. 13, 2020

(54) FLUSHING TUBE FOR ENDOSCOPE AND ENDOSCOPE FLUSHING APPARATUS

(71) Applicant: OLYMPUS CORPORATION, Tokyo (JP)

(72) Inventor: Tatsuya ISHIZUKA, Tokyo (JP)

(73) Assignee: OLYMPUS CORPORATION, Tokyo (JP)

(21) Appl. No.: 16/590,480

(22) Filed: Oct. 2, 2019

Related U.S. Application Data
(63) Continuation of application No. PCT/JP2018/010283, filed on Mar. 15, 2018.

(30) Foreign Application Priority Data
Apr. 4, 2017 (JP) 2017-074529

(51) Int. Cl. A61B 1/12; B08B 9/032

(52) U.S. Cl. CPC A61B 1/125; B08B 2209/032; B08B 9/0321

(57) ABSTRACT

A flushing tube for endoscope includes a mounting section configured to be attached to a liquid feeding device, an endoscope connection section configured to be connected to an endoscope, a tubular portion interconnecting the mounting section and the endoscope connection section, an expansion section arranged between the mounting section and the tubular portion, between the endoscope connection section and the tubular portion, or in at least a portion of the tubular portion and configured to be expanded in response to a rise in an internal pressure, and a cover section arranged at an outer circumference of the expansion section and having a color different than a color of the outer circumference of the expansion section, where the cover section causes the outer circumference of the expansion section to be exposed when the expansion section is placed in an expanded state.

### CROSS REFERENCE TO RELATED APPLICATION

This application is a continuation application of PCT/JP2018/010283 filed on Mar. 15, 2018 and claims benefit of Japanese Application No. 2017-074529 filed in Japan on Apr. 4, 2017, the entire contents of which are incorporated herein by this reference.

### BACKGROUND OF THE INVENTION

### 1. Field of the Invention

The present invention relates to a flushing tube for endoscope that is connected to a conduit of an endoscope and an endoscope flushing apparatus.

### 2. Description of the Related Art

As illustrated in FIG. 1 of Japanese Patent Application Laid-Open Publication No. 2012-50707, a liquid feeding device is known which feeds liquid into a conduit of an endoscope. While the intended use of the liquid feeding device disclosed in Japanese Patent Application Laid-Open Publication No. 2012-50707 is feeding of liquid to a distal end portion of an endoscope that is being used in observation of a living body, the same or similar techniques are also applied to endoscope flushing apparatuses used in cleaning an inside of a conduit of an endoscope that has been used. Cleaning of the inside of the conduit of the endoscope using an endoscope flushing apparatus is performed in a state where the endoscope is placed in a sink or a container such as a tub.

### SUMMARY OF THE INVENTION

A flushing tube for endoscope according to an aspect of the present invention includes a mounting section configured to be attached to a liquid feeding device; an endoscope connection section configured to be connected to an endoscope; a tubular portion interconnecting the mounting section and the endoscope connection section; an expansion section arranged between the mounting section and the tubular portion, between the endoscope connection section and the tubular portion, or in at least a portion of the tubular portion and configured to be expanded in response to a rise in an internal pressure; and a cover section arranged at an outer circumference of the expansion section and having a color different than a color of the outer circumference of the expansion section, where the cover section causes the outer circumference of the expansion section to be exposed when the expansion section is placed in an expanded state. An endoscope flushing apparatus according to an aspect of the present invention also includes a pump to which the mounting section of the flushing tube for endoscope is attached, the pump being configured to transfer liquid from the mounting section to the endoscope connection section.

### BRIEF DESCRIPTION OF THE DRAWINGS

FIG. 1 is a diagram illustrating an endoscope flushing apparatus and a flushing tube for endoscope of an embodiment;

FIG. 2 is a diagram illustrating a state where the flushing tube for endoscope is removed from the endoscope flushing apparatus of a first embodiment;

FIG. 3 is an enlarged view of an expansion section of the first embodiment;

FIG. 4 is a cross-sectional view taken along the line IV-IV of FIG. 3;

FIG. 5 is an enlarged view illustrating a state where the expansion section of the first embodiment is expanded;

FIG. 6 is a cross-sectional view taken along the line VI-VI of FIG. 5;

FIG. 7 is a diagram illustrating a modified example of the flushing tube for endoscope of the first embodiment;

FIG. 8 is a diagram illustrating an endoscope flushing apparatus and a flushing tube for endoscope of a second embodiment;

FIG. 9 is a diagram illustrating a modified example of the flushing tube for endoscope of the second embodiment;

FIG. 10 is a diagram illustrating an expansion section and an indicator of a flushing tube for endoscope of a third embodiment;

FIG. 11 is a diagram illustrating the expansion section and the indicator of the flushing tube for endoscope of the third embodiment;

FIG. 12 is a diagram illustrating an expansion section and an indicator of a flushing tube for endoscope of a fourth embodiment;

FIG. 13 is a diagram illustrating a modified example of the indicator of the fourth embodiment;

FIG. 14 is a diagram illustrating an expansion section and an indicator of a flushing tube for endoscope of a fifth embodiment;

FIG. 15 is a diagram illustrating an expansion section and an indicator of the flushing tube for endoscope of the fifth embodiment; and

FIG. 16 is a diagram illustrating an expansion section and an indicator of a flushing tube for endoscope of a sixth embodiment.

### DETAILED DESCRIPTION OF THE PREFERRED EMBODIMENTS

Exemplary modes of the present invention will be described hereinbelow with reference to the drawings. Note that, in the individual figures that are used for the following explanations, scales of the individual components are varied so that the individual components have sizes and dimensions recognizable on the figures. The present invention is not limited to the number and quantity of the components illustrated in these figures, the shapes of the components, the ratios between the sizes of the components, and the relative positional relationships between the individual components.

### First Embodiment

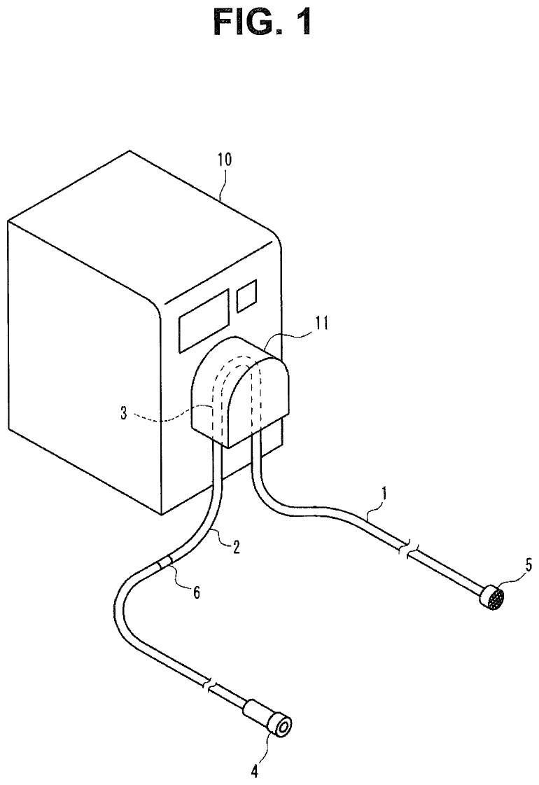

FIG. 1 illustrates an endoscope flushing apparatus 10 and a flushing tube for endoscope 1 of this embodiment. The endoscope flushing apparatus 10 is a device that feeds liquid into a conduit provided in a not-shown endoscope and cleans the inside of the conduit.

The endoscope flushing apparatus 10 includes the flushing tube for endoscope 1 connected to the conduit of the endoscope and a pump 11 that feeds the liquid into the conduit via the flushing tube for endoscope 1.

While details will be described later, the flushing tube for endoscope 1 includes an endoscope connection section 4 configured to be connected to the conduit of the endoscope, a mounting section 3 configured to be attached to the endoscope flushing apparatus 10, and a tubular portion 2 interconnecting the endoscope connection section 4 and the mounting section 3. Although not shown, the endoscope flushing apparatus 10 includes a motor that drives the pump 11 and a power-supply device.

The endoscope flushing apparatus 10 may be configured to include a container that stores part or all of the liquid to be fed into the conduit of the endoscope. It may also be configured to not include such a container. In the case where the endoscope flushing apparatus 10 does not include a container for storing the liquid, the endoscope flushing apparatus 10 includes a feature for pumping out the liquid stored in another container by the pump 11 and feeding the liquid into the conduit of the endoscope.

In this embodiment, as an example, the endoscope flushing apparatus 10 pumps out the liquid out of a container such as a sink and a tub storing the liquid and feeds the liquid into the conduit of the endoscope. In this embodiment, in the container in which the liquid is stored, the endoscope is arranged in a state where the endoscope is immersed in the liquid. Accordingly, in this embodiment, during the operation of the pump 11, the liquid in the container after having been pumped out of the container flows such that the liquid passes through the conduit of the endoscope and returns to the inside of the container.

The type of the liquid that the endoscope flushing apparatus 10 feeds into the conduit of the endoscope is not particularly limited. The liquid fed by the endoscope flushing apparatus 10 may be water or a liquid mixture of cleaning liquid and water. The endoscope flushing apparatus 10 may feed not only liquid but also air. When air is fed by the endoscope flushing apparatus 10, it is made possible to evacuate the water remaining in the conduit of the endoscope therefrom.

The configuration of the pump 11 is not particularly limited. In this embodiment, as an example, the pump 11 is a roller pump. The roller pump is also called a tube pump or a peristaltic pump. The roller pump includes a roller that compresses part of the tube made of a material having elasticity, and the fluid in the tube is made to move by the location at which the part of the tube is compressed by the roller being moved in the longitudinal direction of the tube. Since the configuration of the roller pump is known, detailed explanations thereof are omitted. Note that the configuration of the pump 11 may be any other form.

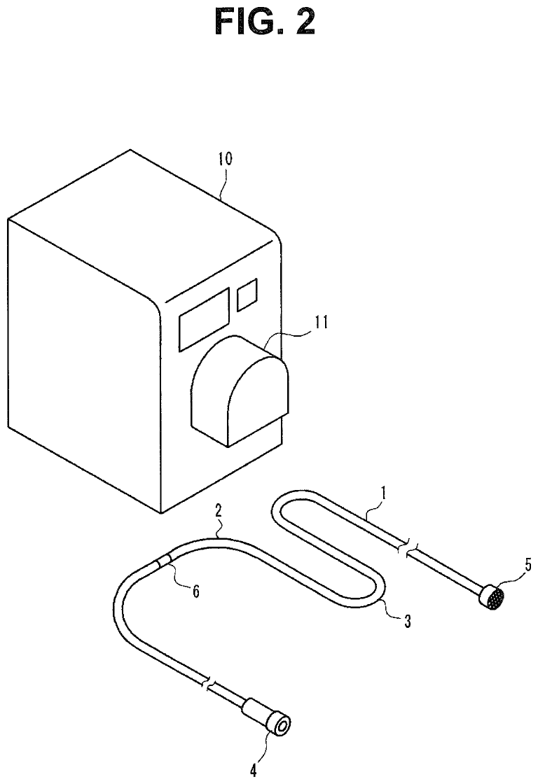

In this embodiment, as an example, the pump 11 is configured such that the roller directly compresses the tubular mounting section 3 which is a part of the flushing tube for endoscope 1. In other words, the mounting section 3 of the flushing tube for endoscope 1 is a region that is compressed by the pump 11 which is a roller pump. The flushing tube for endoscope 1 is, as illustrated in FIG. 2, removable from the pump 11.

The flushing tube for endoscope 1 of this embodiment includes a tubular portion 2 made of resin having elasticity. The mounting section 3 is part of the tubular portion 2. While it is possible to define any appropriate position of the tubular portion 2 as the mounting section 3, in this embodiment, an intermediate portion of the tubular portion 2 corresponds to the mounting section 3. Note that, on the outer surface of the tubular portion 2, an indicator indicative of the position of the mounting section 3 may be provided, where the indicator may be provided by being printed thereon or as a sticker, etc.

The endoscope connection section 4 configured to be connected to the conduit of the endoscope is provided at one end of the tubular portion 2. The endoscope connection section 4 includes a connector configured to be connectable to a pipe sleeve at which the conduit of the endoscope has an opening. Note that the endoscope connection section 4 may be configured to be connectable to a plurality of conduits which the endoscope may include.

A suction section 5 is provided at the other end of the tubular portion 2. The suction section 5 includes an opening through which the liquid stored in the container is sucked into the tubular portion 2. The suction section 5 may be simply an end of the tubular portion 2 that has been cut or it may include a filter for filtering of the water that is being sucked.

As has been described in the foregoing, in this embodiment, the pump 11 has the configuration of a roller pump that directly compresses the tubular portion 2, and the suction section 5 and the endoscope connection section 4 are interconnected using one single tubular portion 2. Accordingly, in this embodiment, a connector that interconnects the pump 11 and the flushing tube for endoscope 1 does not need to be provided. In this embodiment, the flushing tube for endoscope 1 can be configured using one single tubular portion 2. Accordingly, in this embodiment, the number of components that constitute the endoscope flushing apparatus 10 and the flushing tube for endoscope 1 can be reduced.

Next, the details of the features of the flushing tube for endoscope 1 will be described. The flushing tube for endoscope 1 includes an expansion section 6 and an indicator 7 in addition to the endoscope connection section 4, the mounting section 3, and the tubular portion 2 which have been described in the foregoing.

The expansion section 6 is arranged in at least a part of the section between the mounting section 3 and the endoscope connection section 4 of the tubular portion 2, and configured to be expanded when an internal pressure becomes equal to or larger than a predetermined value. In other words, the expansion section 6 is arranged on the downstream side relative to the pump 11 when the endoscope flushing apparatus 10 is used.

The expansion section 6 is made of a material such as resin having elasticity and has an internal space 6a that is in communication with the inside of the tubular portion 2. The pressure in the internal space 6a of the expansion section 6 becomes equal to the pressure in the section between the mounting section 3 and the endoscope connection section 4 of the tubular portion 2.

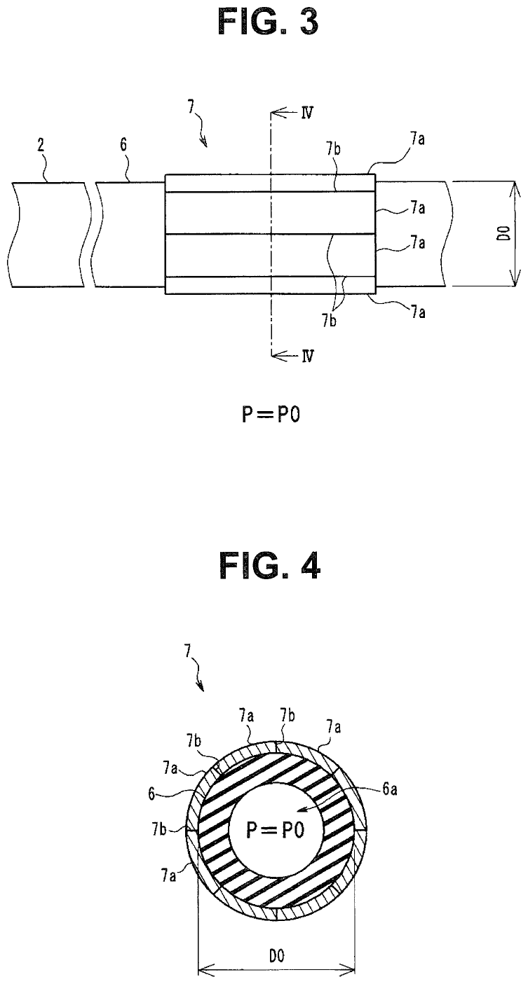

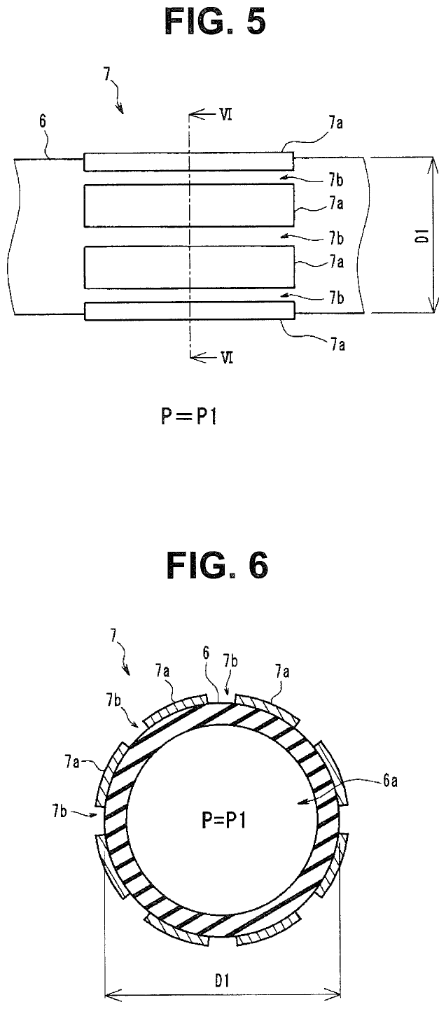

FIG. 3 is a diagram in which the expansion section 6 in a state where the internal pressure is equal to the reference pressure P0 is illustrated in an enlarged state. FIG. 4 is a cross-sectional view taken along the line IV-IV of FIG. 3. FIG. 5 is a diagram that illustrates the expansion section 6 in a state where the internal pressure is equal to or larger than a predetermined value. FIG. 6 is a cross-sectional view taken along the line VI-VI of FIG. 5.

In this embodiment, as an example, the expansion section 6 is configured by at least a part of the section between the mounting section 3 and the endoscope connection section 4 of the tubular portion 2. In other words, as illustrated in FIGS. 3 and 4, the expansion section 6 of this embodiment has a tubular shape. When the internal pressure of the expansion section 6 and the tubular portion 2 is equal to an atmospheric pressure, then the outer diameters of the expansion section 6 and the tubular portion 2 are equal to each other. Note that the tubular portion 2 as a whole may constitute the expansion section 6.

While the position of the expansion section 6 is not particularly limited, at least a part of the expansion section 6 is preferably exposed above the surface of the liquid in a state where the endoscope connection section 4 is connected to the conduit of the endoscope immersed in the liquid in the container.

Here, the pressure P of the internal space 6a of the expansion section 6 in a case where the pump 11 is operated with nothing connected to the endoscope connection section 4 is defined as a reference pressure P0. In addition, the outer diameter of the expansion section 6 in a case where the pressure P of the internal space 6a is equal to the reference pressure P0 is defined as D0.

The expansion section 6 is expanded such that the outer diameter becomes equal to or larger than D1 which is larger than D0 in a case where the pressure P of the internal space 6a is equal to a predetermined pressure P1 which is higher than the reference pressure P0. The pressure P1 is, for example, the pressure of the internal space 6a of the expansion section 6 in the case where the pump 11 is operated in a state where narrowing occurs in the conduit of the endoscope connected to the endoscope connection section 4.

The ratio between the outer diameter D0 and the outer diameter D1 is not particularly limited. In this embodiment, as an example, the outer diameter D1 of the expansion section 6 at the time of expansion is more than 1.2 times larger than the outer diameter D0 in a case where the pressure P is equal to the reference pressure P0.

The indicator 7 is provided on the outer circumference of the expansion section 6. The indicator 7 includes a plurality of cover sections 7a arranged on the outer circumference of the expansion section 6. The cover sections 7a are arranged on the outer circumference of the expansion section 6 in a state where a slit 7b which is a cut is provided therebetween. The individual cover sections 7a have a film-like shape or a thin plate-like shape. The color of the outer circumference of the cover section 7a is different from the color of the outer circumference of the expansion section 6.

While the configuration of the cover section 7a is not particularly limited, in this embodiment, as an example, the cover section 7a is configured by an ink added on the outer circumference of the expansion section 6 using a printing technique. Note that the cover section 7a may be a sheet-like component adhered to the outer circumference of the expansion section 6 using an adhesive.

In this embodiment, as an example, the expansion section 6 is made of white translucent resin, and the color of the outer circumference of the cover section 7a is, for example, black or gray.

The cover sections 7a of this embodiment are arranged on the entire expansion section 6 in its circumferential direction so as to be arranged in the circumferential direction of the outer circumference of the tubular expansion section 6. In this embodiment, the slits 7b provided between the cover sections 7a have a width that causes difficulty for a naked eye of a person to visually recognize them in a case where the outer diameter of the expansion section 6 is equal to D0. In other words, in a case where the outer diameter of the expansion section 6 is equal to D0, the cover sections 7a will be seen as one single continuous film covering the expansion section 6.

Note that the specific numerical value of the width of the slit 7b in the case where the outer diameter of the expansion section 6 is equal to D0 is not particularly limited. The width of the slit 7b in the case where the outer diameter of the expansion section 6 is equal to D0 should be defined such that, for example, visual recognition thereof from a position away from the indicator 7 by 0.5 meter or larger is difficult. The width of the slit 7b in the case where the outer diameter of the expansion section 6 is equal to D0 may be zero.

In addition, when the outer diameter of the expansion section 6 is expanded to D1, as illustrated in FIGS. 5 and 6, the width of the slit 7b is enlarged, and the slit 7b will have a width that facilitates visibility to a naked eye of a person. In other words, the width of the slit 7b is enlarged such that the outer circumference of the expansion section 6 is exposed to the outside in a case where the internal pressure of the expansion section 6 is equal to or larger than the predetermined value P1.

Accordingly, in the flushing tube for endoscope 1 of this embodiment, in the case where the internal pressure of the expansion section 6 is equal to or larger than the predetermined value P1, the outer circumference of the expansion section 6 having a color different than that of the cover section 7a can be seen via the slit 7b between the cover sections 7a.

When the inside of the conduit of the endoscope should be cleaned using the endoscope flushing apparatus 10 and the flushing tube for endoscope 1 having the above-described features, first, the mounting section 3 of the flushing tube for endoscope 1 is attached to the endoscope flushing apparatus 10 and the endoscope connection section 4 is connected to the conduit of the endoscope. In addition, the suction section 5 of the endoscope and the flushing tube for endoscope 1 is immersed in the liquid stored in the container, and the operation of the pump 11 is started.

By the above-described operation, the liquid stored in the container is fed into the conduit of the endoscope after it has been sucked out of the suction section 5. At this point, if no clogging such as narrowing and blocking occurs in the conduit of the endoscope, then the flow resistance of the conduit is low and the pressure P of the internal space 6a of the expansion section 6 will be substantially equal to the reference pressure P0. On the other hand, if clogging such as narrowing and blocking occurs in the conduit of the endoscope, the pressure P of the internal space 6a of the expansion section 6 will be equal to or larger than the predetermined pressure P1.

In other words, in the endoscope flushing apparatus 10 and the flushing tube for endoscope 1 of this embodiment, during the execution of the operation to feed the liquid into the conduit of the endoscope, if no clogging occurs in the conduit of the endoscope, the width of the slit 7b is narrow, and the outer circumference of the expansion section 6 cannot be visually recognized at the indicator 7. On the other hand, in this embodiment, if clogging occurs in the conduit of the endoscope, the width of the slit 7b is enlarged, and the outer circumference of the expansion section 6 will be visually recognizable at the indicator 7 between the cover sections 7a.

Since the color of the cover section 7a differs from the color of the outer circumference of the expansion section 6, whether or not the outer circumference of the expansion section 6 can be seen via the slit 7b between the cover sections 7a can be readily determined by a user.

Accordingly, in accordance with the endoscope flushing apparatus 10 and the flushing tube for endoscope 1 of this embodiment, presence or absence of clogging in the conduit of the endoscope can be confirmed by visually recognizing, at the indicator 7, whether or not the outer circumference of the expansion section 6 is exposed.

The endoscope flushing apparatus 10 and the flushing tube for endoscope 1 of this embodiment can be configured in an inexpensive manner, for presence or absence of clogging in the conduit of the endoscope can be confirmed without using an electronic circuit that includes a pressure sensor, a flow sensor, and the like.

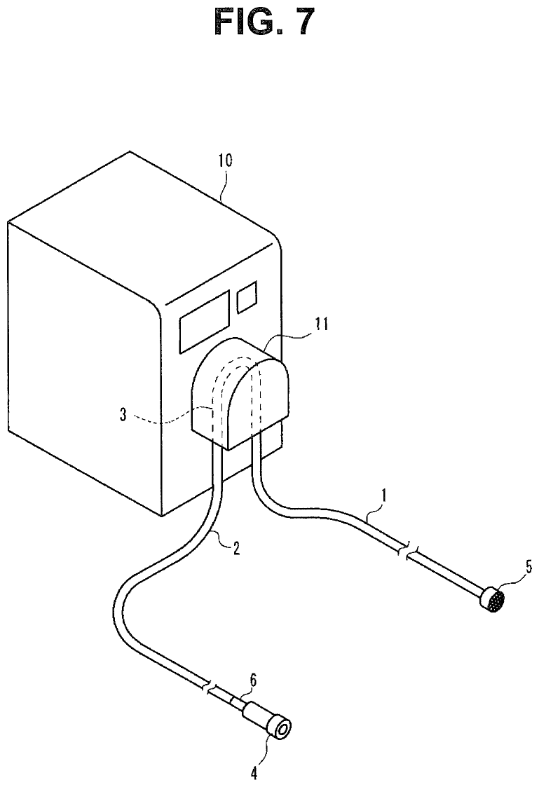

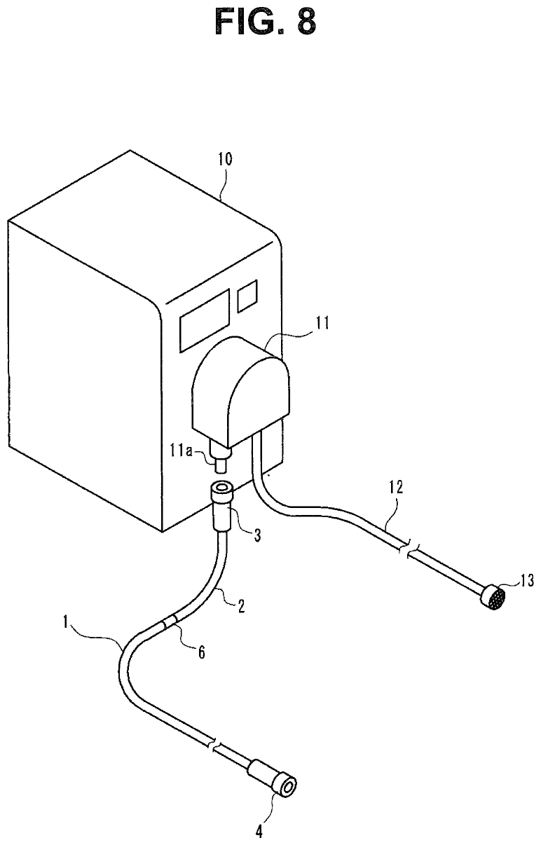

Note that although the expansion section 6 is provided in the tubular portion 2 in this embodiment, the expansion section 6 may be provided between the endoscope connection section 4 and the tubular portion 2 as illustrated in FIG. 7 as a modified example.

### Second Embodiment

A second embodiment of the present invention will be described hereinbelow. Note that only the aspects different than those in the first embodiment will be described below, the components identical or similar to those in the first embodiment are denoted by the same reference signs, and description thereof will be omitted as appropriate.

FIG. 7 is a diagram that illustrates the endoscope flushing apparatus 10 and the flushing tube for endoscope 1 of the second embodiment. While the sections from the suction section that sucks the liquid out of the container to the endoscope connection section are interconnected using one single tubular portion in the first embodiment, the same portion is divided into two tubular portions on the front and rear sides of the pump 11 in this modified example.

In other words, the endoscope flushing apparatus 10 of this embodiment includes, at one end thereof, a suction section 13 that sucks the liquid out of the container, and, at the other end, a suction tube 12 configured to be connected to the pump 11.

The endoscope flushing apparatus 10 of this embodiment also includes a discharge connector 11 a which is a port from which the liquid is discharged during the operation of the pump 11. In addition, the mounting section 3 of the flushing tube for endoscope 1 of this embodiment, which is provided at the end opposite to the end at which the endoscope connection section 4 of the tubular portion 2 resides, is a connector configured to be connectable to the discharge connector 11a.

This embodiment that has been described above differs from the first embodiment only in the feature for attaching the flushing tube for endoscope 1 to the endoscope flushing apparatus 10. Accordingly, in the endoscope flushing apparatus 10 and the flushing tube for endoscope 1 of this embodiment as well, in the same manner as in the first embodiment, if no clogging occurs in the conduit of the endoscope during the execution of the operation to feed the liquid into the conduit of the endoscope, the width of the slit 7b is narrow and the outer circumference of the expansion section 6 cannot be visually recognized at the indicator 7. On the other hand, in this embodiment, if clogging occurs in the conduit of the endoscope, the width of the slit 7b is enlarged and the outer circumference of the expansion section 6 will be visually recognizable at the indicator 7 between the cover sections 7a.

Accordingly, in accordance with the endoscope flushing apparatus 10 and the flushing tube for endoscope 1 of this embodiment, presence or absence of clogging in the conduit of the endoscope can be confirmed by visually recognizing, at the indicator 7, whether or not the outer circumference of the expansion section 6 is exposed.

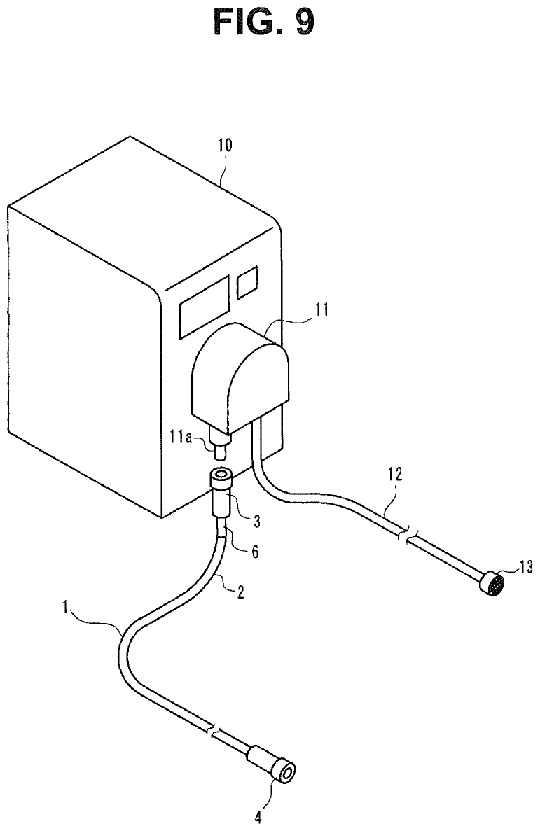

Note that, in this embodiment as well as in the modified example of the first embodiment illustrated in FIG. 7, the expansion section 6 may be provided between the endoscope connection section 4 and the tubular portion 2. The expansion section 6 may also be provided between the mounting section 3 and the tubular portion 2 as illustrated as the modified example of this embodiment illustrated in FIG. 9.

### Third Embodiment

A third embodiment of the present invention will be described hereinbelow. Note that only the aspects different than those in the first and second embodiments will be described below, the components identical or similar to those in the first and second embodiments are denoted by the same reference signs, and description thereof will be omitted as appropriate.

This embodiment differs from the first embodiment in its configuration of the indicator 7 of the flushing tube for endoscope 1. In the above-described first embodiment, the cover sections 7a of the indicator 7 are arranged in the circumferential direction on the outer circumference of the tubular expansion section 6, but the mode of arrangement of the cover sections 7a is not limited to the first embodiment.

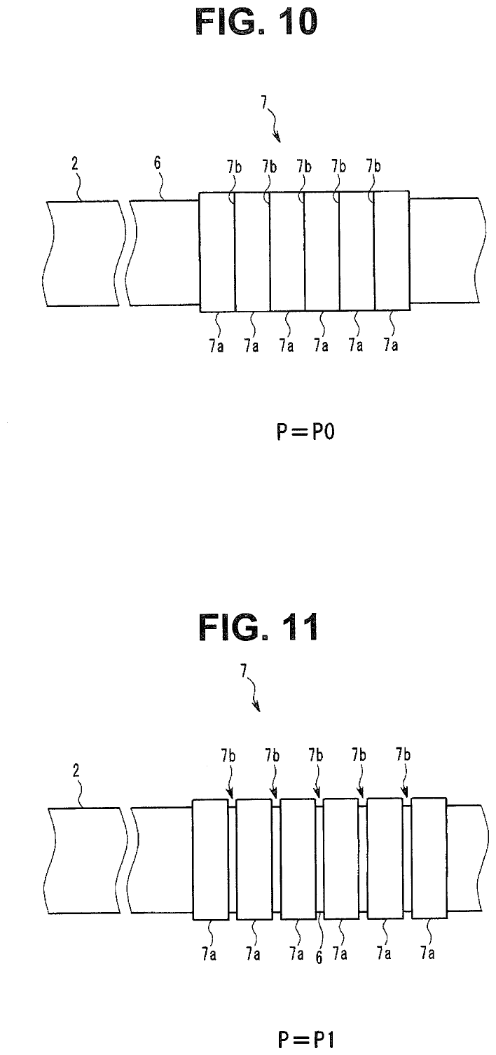

As illustrated in FIGS. 10 and 11, the cover sections 7a of the indicator 7 of this embodiment are arranged on the outer circumference of the expansion section 6 so as to be arranged in the longitudinal direction of the expansion section 6. The expansion section 6 is expanded not only in the radial direction but also in the longitudinal direction in response to the rise in the internal pressure.

A slit 7b is provided between the cover sections 7a. In the same manner as in the first embodiment, if the internal pressure of the expansion section 6 is equal to the reference pressure P0, the slit 7b has a width that causes difficulty for a naked eye of a person to visually recognize it. Accordingly, if the internal pressure of the expansion section 6 is equal to the reference pressure P0, the cover sections 7a will be seen as one single continuous film that covers the expansion section 6 as illustrated in FIG. 10. If the internal pressure of the expansion section 6 is equal to the predetermined pressure P1 which is higher than in the case where it is equal to the reference pressure P0, the expansion section 6 is expanded, and the width of the slit 7b is enlarged until the width of the slit 7b becomes visually recognizable as illustrated in FIG. 11.

As has been described in the foregoing, in the endoscope flushing apparatus 10 and the flushing tube for endoscope 1 of this embodiment, if no clogging occurs in the conduit of the endoscope during the execution of the operation to feed the liquid into the conduit of the endoscope, the width of the slit 7b is narrow and the outer circumference of the expansion section 6 cannot be visually recognized at the indicator 7. On the other hand, in this embodiment, if clogging occurs in the conduit of the endoscope, the width of the slit 7b is enlarged and the outer circumference of the expansion section 6 will be visually recognizable at the indicator 7 between the cover sections 7a.

Accordingly, in accordance with the endoscope flushing apparatus 10 and the flushing tube for endoscope 1 of this embodiment, presence or absence of clogging in the conduit of the endoscope can be continued by visually recognizing, at the indicator 7, whether or not the outer circumference of the expansion section 6 is exposed.

Note that, in this embodiment as well as in the modified example of the first embodiment illustrated in FIG. 7, the expansion section 6 may be provided between the endoscope connection section 4 and the tubular portion 2. The expansion section 6 may also be provided between the mounting section 3 and the tubular portion 2 as in the modified example of the second embodiment illustrated in FIG. 9.

### Fourth Embodiment

A fourth embodiment of the present invention will be described hereinbelow. Note that only the aspects different than those in the third embodiment will be described below, the components identical or similar to those in the third embodiment are denoted by the same reference signs, and description thereof will be omitted as appropriate.

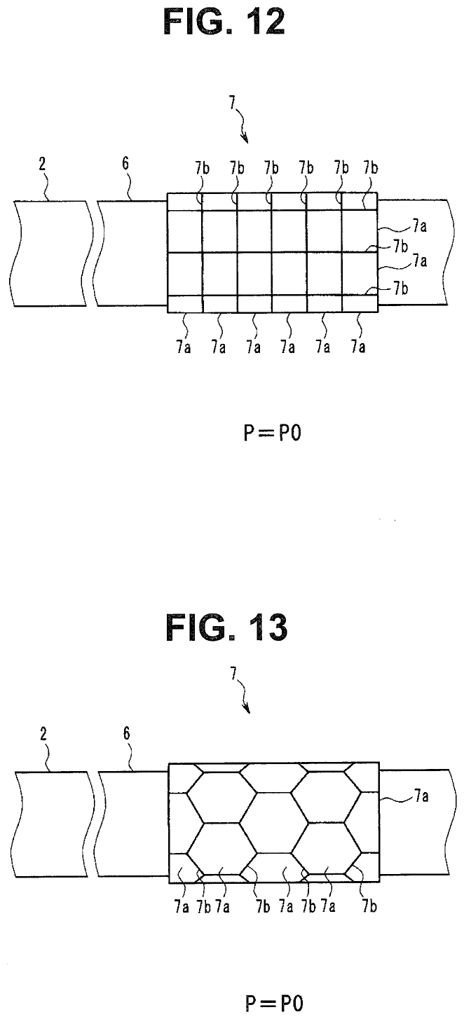

This embodiment differs from the third embodiment in its configuration of the indicator 7 of the flushing tube for endoscope 1. As illustrated in FIG. 12, the cover sections 7a of the indicator 7 of this embodiment are arranged on the outer circumference of the expansion section 6 in the circumferential direction and the longitudinal direction. In other words, the slit 7b has a mesh-like shape. The expansion section 6 is expanded in the radial direction and the longitudinal direction in response to the rise in the internal pressure.

The slit 7b has, in the same manner as in the third embodiment, a width that causes difficulty for a naked eye of a person to visually recognize it if the internal pressure of the expansion section 6 is equal to the reference pressure P0. Also, if the internal pressure of the expansion section 6 is equal to the predetermined pressure P1 which is higher than in the case where it is equal to the reference pressure P0, the expansion section 6 is expanded and the width of the slit 7b is enlarged until the width of the slit 7b becomes visually recognizable.

In the endoscope flushing apparatus 10 and the flushing tube for endoscope 1 of this embodiment, if no clogging occurs in the conduit of the endoscope during the execution of the operation to feed the liquid into the conduit of the endoscope, the width of the slit 7b is narrow and the outer circumference of the expansion section 6 cannot be visually recognized at the indicator 7. On the other hand, in this embodiment, if clogging occurs in the conduit of the endoscope, the width of the slit 7b is enlarged and the outer circumference of the expansion section 6 will be visually recognizable at the indicator 7 between the cover sections 7a.

Accordingly, in accordance with the endoscope flushing apparatus 10 and the flushing tube for endoscope 1 of this embodiment, presence or absence of clogging in the conduit of the endoscope can be confirmed by visually recognizing, at the indicator 7, whether or not the outer circumference of the expansion section 6 is exposed.

Note that, in this embodiment as well as in the modified example of the first embodiment illustrated in FIG. 7, the expansion section 6 may be provided between the endoscope connection section 4 and the tubular portion 2. The expansion section 6 may also be provided between the mounting section 3 and the tubular portion 2 as in the modified example of the second embodiment illustrated in FIG. 9.

The shape of the individual cover sections 7a is not limited to a parallelogram, and, as illustrated in FIG. 13, the shape of the individual cover sections 7a may be hexagonal. In this modified example, the slit 7b has a honeycomb shape.

### Fifth Embodiment

A fifth embodiment of the present invention will be described hereinbelow. Note that only the aspects different than those in the first and second embodiments will be described below, the components identical or similar to those in the first and second embodiments are denoted by the same reference signs, and description thereof will be omitted as appropriate.

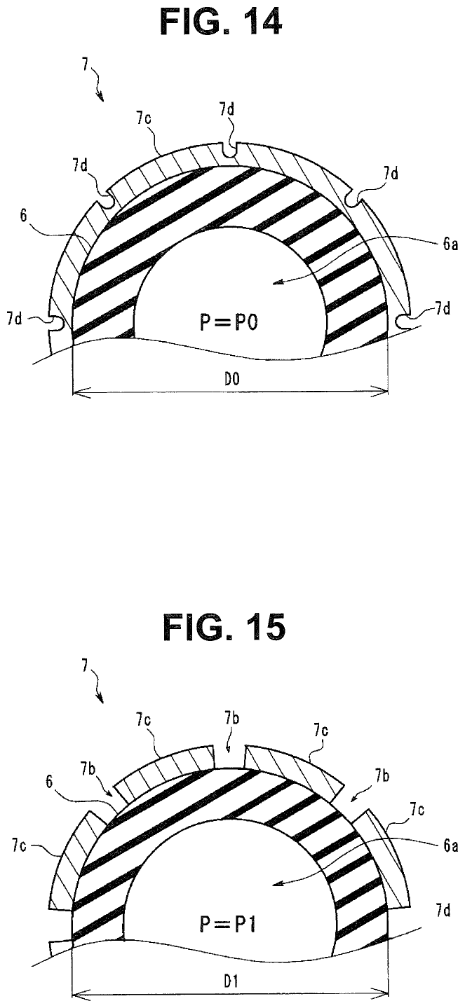

This embodiment differs from the first embodiment in its configuration of the indicator 7 of the flushing tube for endoscope 1. The indicator 7 of this embodiment illustrated in FIGS. 14 and 15 is arranged on the outer circumference of the expansion section 6, and includes one or more cover sections 7c having a color different than that of the outer circumference of the expansion section 6 and a split groove 7d provided in the cover section 7c.

The cover section 7c has a film-like shape or a thin plate-like shape. The cover section 7c of this embodiment covers the entire expansion section 6 in the circumferential direction so as to be arranged in the circumferential direction of the outer circumference of the tubular expansion section 6. While the configuration of the cover section 7c is not particularly limited, in this embodiment, as an example, the cover section 7c is configured by an ink added on the outer circumference of the expansion section 6 using a printing technique. Note that the cover section 7c may be a sheet-like component adhered to the outer circumference of the expansion section 6 using an adhesive.

In this embodiment, as an example, the expansion section 6 is made of white translucent resin, and the color of the outer circumference of the cover section 7c is, for example, black or gray.

The split groove 7d is provided to form a thin portion at which the cover section 7c is torn apart when the expansion section 6 is expanded and the surface area of the outer circumference of the expansion section 6 is increased. The cross-sectional shape of the split groove 7d is not particularly limited as long as it promotes the break of the cover section 7c. The split groove 7d may be a V-shaped groove. The split groove 7d may also have a perforation-like shape penetrating partly the cover section 7c.

In this embodiment, if the internal pressure of the expansion section 6 is equal to the reference pressure P0, the cover section 7c is in a connected state as illustrated in FIG. 14. If the internal pressure of the expansion section 6 is equal to the predetermined pressure P1 which is higher than in the case where it is equal to the reference pressure P0, the expansion section 6 is expanded as illustrated in FIG. 15 and the cover section 7c is torn apart at the portion of the split groove 7d and split into multiple pieces. Accordingly, in this embodiment, when the expansion section 6 is expanded, then the slit 7b is created which causes the outer circumference of the expansion section 6 to be exposed at the indicator 7.

According to the endoscope flushing apparatus 10 and the flushing tube for endoscope 1 of this embodiment that has been described in the foregoing, in the same manner as in the first and second embodiments, presence or absence of clogging in the conduit of the endoscope can be confirmed by visually recognizing, at the indicator 7, whether or not the outer circumference of the expansion section 6 is exposed.

Note that, in this embodiment as well as in the modified example of the first embodiment illustrated in FIG. 7, the expansion section 6 may be provided between the endoscope connection section 4 and the tubular portion 2. The expansion section 6 may also be provided between the mounting section 3 and the tubular portion 2 as in the modified example of the second embodiment illustrated in FIG. 9.

The split groove 7d of this embodiment may be provided as in the first embodiment such that the cover section 7c is made to be split into multiple pieces in the circumferential direction or may be provided as in the third embodiment such that the cover section 7c is made to be split into multiple pieces in the longitudinal direction. The split groove 7d of this embodiment may also be provided as in the fourth embodiment such that the cover section 7c is made to be split in the circumferential direction and the longitudinal direction.

### Sixth Embodiment

The sixth embodiment of the present invention will be described hereinbelow. Note that only the aspects different than those in the first and second embodiments will be described below, the components identical or similar to those in the first and second embodiments are denoted by the same reference signs, and description thereof will be omitted as appropriate.

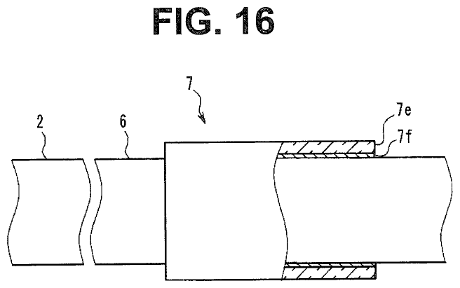

The indicator 7 of the flushing tube for endoscope 1 of this embodiment illustrated in FIG. 16 includes a transparent tube 7e that covers the outer circumference of the expansion section 6 and a pressure sensitive layer 7f provided between the expansion section 6 and the transparent tube 7e.

The transparent tube 7e is made of transparent or translucent resin. The user can visually recognize the pressure sensitive layer 7f from the outer side of the transparent tube 7e. The transparent tube 7e has a higher rigidity than the expansion section 6. The inner diameter of the transparent tube 7e is equal to the outer diameter D0 of the expansion section 6 in the case where the internal pressure of the expansion section 6 is equal to the reference pressure P0.

The color of the outer circumference of the pressure sensitive layer 7f changes when it is pressed against the inner circumference of the transparent tube 7e with a predetermined pressure. With regard to the pressure sensitive layer 7f, for example, techniques similar to those of carbonless copy paper can be used.

In this embodiment, when the expansion section 6 is expanded, the pressure sensitive layer 7f is pressed against the inner circumference of the transparent tube 7e by the outer circumference of the expansion section 6.

If the internal pressure of the expansion section 6 is equal to the reference pressure P0, the pressure applied to the pressure sensitive layer 7f by the expansion section 6 is small, and the color of the pressure sensitive layer 7f does not change. The color of the pressure sensitive layer 7f changes if the internal pressure of the expansion section 6 is equal to the predetermined pressure P1 which is higher than in the case where it is equal to the reference pressure P0.

In the endoscope flushing apparatus 10 and the flushing tube for endoscope 1 of this embodiment, the color of the pressure sensitive layer 7f does not change if no clogging occurs in the conduit of the endoscope during the execution of the operation to feed the liquid into the conduit of the endoscope. On the other hand, in this embodiment, the color of the pressure sensitive layer 7f changes if clogging occurs in the conduit of the endoscope.

Accordingly, in accordance with the endoscope flushing apparatus 10 and the flushing tube for endoscope 1 of this embodiment, presence or absence of clogging in the conduit of the endoscope can be confirmed by visually confirming, at the indicator 7, whether or not the color of the pressure sensitive layer 7f has changed.

Note that, in this embodiment as well as in the modified example of the first embodiment illustrated in FIG. 7, the expansion section 6 may be provided between the endoscope connection section 4 and the tubular portion 2. The expansion section 6 may also be provided between the mounting section 3 and the tubular portion 2 as in the modified example of the second embodiment illustrated in FIG. 9.

The endoscope connection section 4 of the flushing tube for endoscope 1 of the present invention, including the above-described embodiments, may be configured such that the tubular portion end is closed when the endoscope connection section 4 is not connected to an endoscope and the tubular portion end is opened when the endoscope connection section 4 is connected to the endoscope. When it is so configured, the liquid does not flow out from the flushing tube in a case where the endoscope connection section 4 and the endoscope are detached from each other during the execution of the operation to feed the liquid into the conduit of the endoscope by the endoscope flushing apparatus 10 and the flushing tube for endoscope 1, so that the expansion section 6 is expanded. As a consequence, it has an advantage that the user will notice the connection failure. With regard to the opening and closing structure for the tubular portion end, traditionally known techniques can be adopted with modifications made as appropriate.

The present invention is not limited to the above-described embodiments and can be modified as appropriate within a range where such modifications do not contradict the purport or the idea of the invention that can be read from the scope of claims and the specification as a whole, and endoscope flushing apparatuses and flushing tubes for endoscope involving such modifications will also be encompassed by the technical scope of the present invention.

What is claimed is:

1. A flushing tube for endoscope comprising:
a mounting section configured to be attached to a liquid feeding device;
an endoscope connection section configured to be connected to an endoscope;
a tubular portion interconnecting the mounting section and the endoscope connection section;
an expansion section arranged between the mounting section and the tubular portion, between the endoscope connection section and the tubular portion, or in at least a portion of the tubular portion and configured to be expanded in response to a rise in an internal pressure; and
a cover section arranged at an outer circumference of the expansion section and having a color different than a color of the outer circumference of the expansion section,
wherein the cover section causes the outer circumference of the expansion section to be exposed when the expansion section is placed in an expanded state.

2. The flushing tube for endoscope according to claim 1, wherein
a plurality of the cover sections are arranged such that the cover sections are adjacent to each other,
an indicator is provided, the indicator including a slit provided between the cover sections, and
a width of the slit is enlarged such that an outer circumference of the expansion section is exposed to an outside when an internal pressure of the expansion section is equal to or larger than a predetermined value.

3. The flushing tube for endoscope according to claim 1, wherein
the cover section is provided as at least one unit,
an indicator is provided, the indicator including a split groove provided in the cover section, and
the cover section is configured to be broken at a portion where the split groove is provided when an internal pressure of the expansion section is equal to or larger than a predetermined value.

4. The flushing tube for endoscope according to claim 1, wherein an outer diameter of the expansion section when the expansion section is expanded in response to an internal pressure being equal to or larger than a predetermined value is more than 1.2 times larger than an outer diameter of the expansion section when the internal pressure corresponds to an atmospheric pressure and the expansion section is not expanded.

5. An endoscope flushing apparatus comprising a pump to which the mounting section of the flushing tube for endoscope according to claim 1 is attached, the pump being configured to transfer liquid from the mounting section to the endoscope connection section.
